(19) United States
(12) Patent Application Publication
(10) Pub. No.: US 2021/0121160 A1
(43) Pub. Date: Apr. 29, 2021

(54) SYRINGE

(71) Applicant: Medispose, LLC, Lafayette, LA (US)

(72) Inventor: Patrick Thibodaux, Lafayette, LA (US)

(73) Assignee: Medispose, LLC, Lafayette, LA (US)

(21) Appl. No.: 17/080,392

(22) Filed: Oct. 26, 2020

Related U.S. Application Data
(60) Provisional application No. 62/927,519, filed on Oct. 29, 2019.

(51) Int. Cl. A61B 10/00; A61B 10/02

(52) U.S. Cl. CPC A61B 10/0048; A61B 17/4208; A61B 10/0283

(57) ABSTRACT

A syringe operable to rupture the amniotic sac and extract a pure, uncontaminated sample of amniotic fluid. The syringe includes a housing having an open insertion end opposite a handle end. The open insertion end includes a recessed portion with puncture member and fluid opening disposed therein. A plunger disposed within the tubular housing is operable draw the amniotic sac into the recessed portion of the syringe until it contacts the puncture member. The puncture member is operable to pierce the amniotic sac, and the plunger further provides a means for drawing a sample of amniotic fluid through the fluid opening and into the fluid receiving area within the syringe housing.

### RELATED APPLICATIONS

This application claims priority to U.S. Provisional Application No. 62/927,519 filed Oct. 29, 2019. The entire contents of the above application are hereby incorporated by reference as though fully set forth herein.

### FIELD

The present invention relates to a novel method and apparatus of rupturing an amniotic sac and sampling an amniotic fluid. More particularly, the invention relates to a serrated syringe operable to rupture an amniotic sac without endangering a fetus while simultaneously sampling pure, uncontaminated amniotic fluid.

### BACKGROUND

Labor is induced to stimulate contractions of the uterus in an effort to have a vaginal birth. Labor induction may be recommended if the health of the mother or fetus is at risk. However, in special situations, labor is induced for nonmedical reasons, referred to as elective induction, such as living far away from the hospital. Elective induction should not occur before 39 weeks of pregnancy.

Rupturing the amniotic sac can start contractions or make contractions stronger if they have already begun. In the conventional method, a health care professional makes a small hole in the amniotic sac with a special tool, crochet-like long handled hook attached to the forefinger of medical grade glove, as illustrated in FIG. 1, through a procedure referred to as an amniotomy. In the conventional method, the amniotic sac and membranes are punctured with the tool during a vaginal examination, causing the amniotic fluid to flood out of the sac. However, this combines the amniotic fluid with other bodily fluids of the mother, rendering the amniotic fluids untestable. Risks of the conventional method include laceration of the fetus' scalp, which increases intrauterine infection, and induction of fetal pain, which increases movement and thus can cause umbilical cord prolapse, induction of an occult placenta previa, or vasa previa with subsequent maternal hemorrhage. Accordingly, there is a need for a device and method that no only provides a safer method of rupturing the amniotic sac, but also allows a medical professional to extract a pure, uncontaminated sample of amniotic fluid for testing purposes.

### BRIEF SUMMARY OF THE INVENTION

The present invention seeks to meet the needs of the art by providing a syringe that includes a housing having an open insertion end opposite a handle end. The open insertion end includes a recessed portion with puncture member and fluid opening disposed therein. A plunger disposed within the tubular housing is operable draw the amniotic sac into the recessed portion of the syringe until it contacts the puncture member. The puncture member is operable to pierce the amniotic sac, and the plunger further provides the means for drawing the pure, uncontaminated amniotic fluid through the fluid opening and into the fluid receiving area within the syringe housing.

### BRIEF DESCRIPTION OF THE DRAWINGS

The foregoing and other objects, features, and advantages of the invention are apparent from the following detailed description taken in conjunction with the accompanying drawings in which like parts are given like reference numerals and, wherein:

FIG. 1 illustrates a front view of a prior art device for rupturing an amniotic sac.

FIG. 2 illustrates a front right perspective view of a syringe in accordance with embodiments of the present invention.

FIG. 3 illustrates a front left perspective view of a syringe in accordance with embodiments of the present invention.

FIG. 4 illustrates a cross sectional view of a syringe in accordance with embodiments of the present invention.

FIG. 5 illustrates a cross sectional view of a syringe in accordance with embodiments of the present invention.

FIG. 6 illustrates a cross sectional view of a syringe in accordance with embodiments of the present invention.

FIG. 7 illustrates a front view of a syringe in accordance with embodiments of the present invention.

FIG. 8 illustrates a top left perspective view of a syringe in accordance with embodiments of the present invention.

FIG. 9 illustrates a cross sectional view of a syringe in accordance with embodiments of the present invention.

FIG. 10 illustrates a cutaway perspective view of a syringe in accordance with embodiments of the present invention.

FIG. 11 illustrates a method of using the syringe in accordance with embodiments of the present invention.

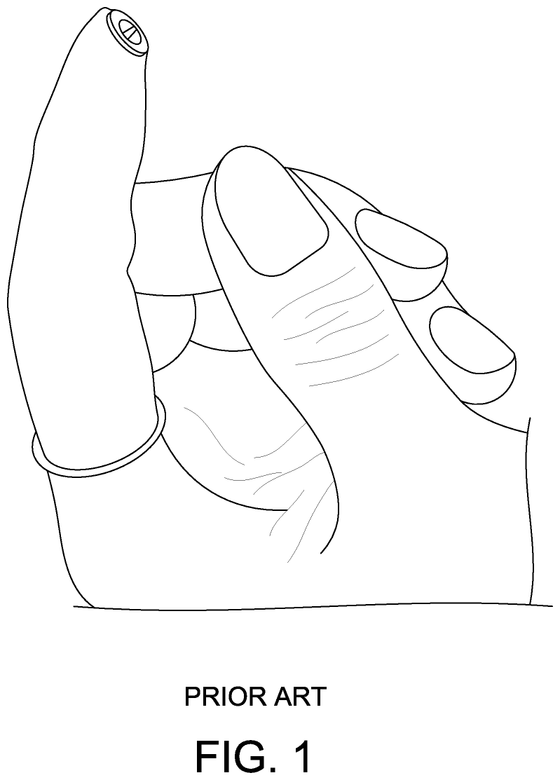

The images in the drawings are simplified for illustrative purposes and are not depicted to scale. Within the descriptions of the figures, similar elements are provided similar names and reference numerals as those of the previous figure(s). The specific numerals assigned to the elements are provided solely to aid in the description and are not meant to imply any limitations (structural or functional) on the invention.

The appended drawings illustrate exemplary configurations of the invention and, as such, should not be considered as limiting the scope of the invention that may admit to other equally effective configurations. It is contemplated that features of one configuration may be beneficially incorporated in other configurations without further recitation.

### DETAILED DESCRIPTION

For a further understanding of the nature and function of the embodiments, reference should be made to the following detailed description. Detailed descriptions of the embodiments are provided herein, as well as, the best mode of carrying out and employing the present invention. It will be readily appreciated that the embodiments are well adapted to carry out and obtain the ends and features mentioned as well as those inherent herein. It is to be understood, however, that the present invention may be embodied in various forms. Therefore, persons of ordinary skill in the art will realize that the following disclosure is illustrative only and not in any way limiting, as the specific details disclosed herein provide a basis for the claims and a representative basis for teaching to employ the present invention in virtually any appropriately detailed system, structure or manner. It should be understood that the devices, materials, methods, procedures, and techniques described herein are presently representative of various embodiments. Other embodiments of the disclosure will readily suggest themselves to such skilled persons having the benefit of this disclosure.

As used herein, “axis” means a real or imaginary straight line about which a three-dimensional body is symmetrical. As used herein, homogeneous is defined as the same in all locations, and a homogeneous material is a material of uniform composition throughout that cannot be mechanically separated into different materials. Examples of “homogeneous materials” are certain types of plastics, ceramics, glass, metals, alloys, paper, board, resins, high-density polyethylene and rubber.

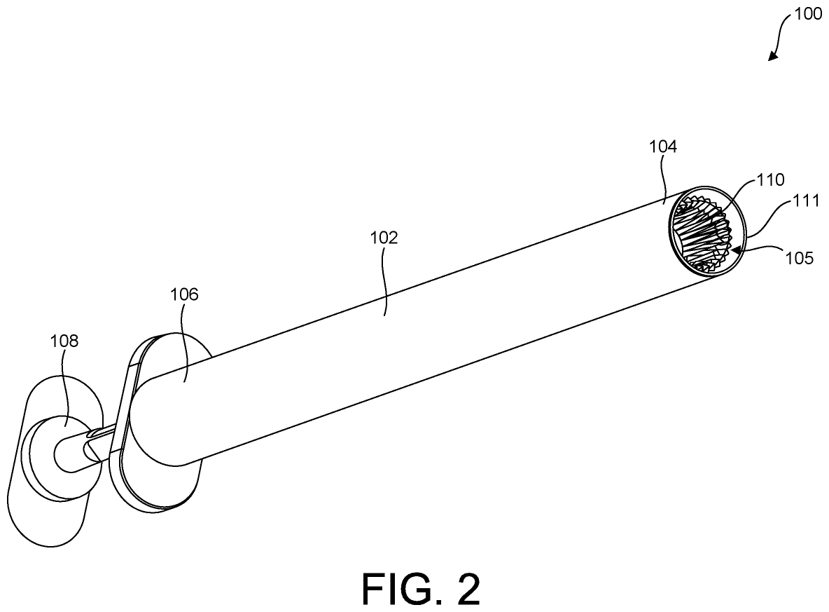

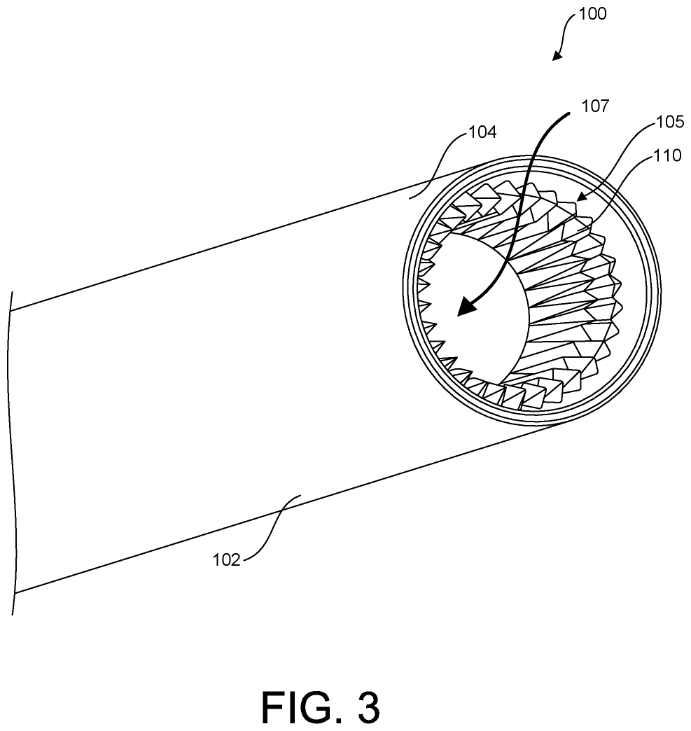

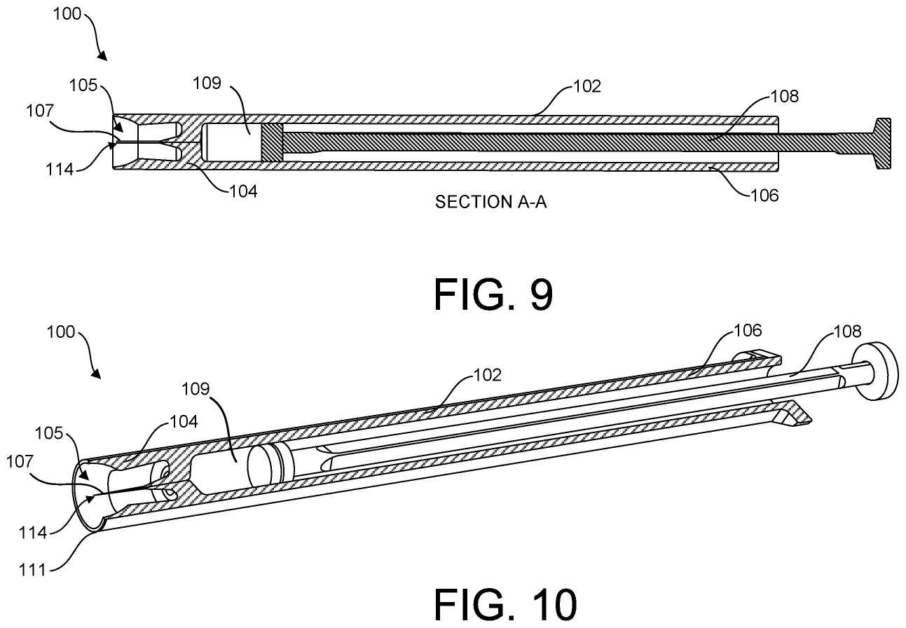

FIGS. 2-10 illustrate a syringe 100 in accordance with embodiments of the invention. The syringe 100 is operable for rupturing an amniotic sac and obtaining a sample of pure, uncontaminated, amniotic fluid. In one embodiment, the syringe 100 includes a tubular housing 102, the housing 102 having an insertion end 104 opposite a handle end 106. The insertion end 104 includes a recessed portion 105 with a fluid opening 107 disposed therein that is in fluid communication with a fluid receiving area 109 in the housing 102. The insertion end 104 is operable for insertion into the vaginal canal of a patient. The handle end 106 is operable for holding by a medical practitioner. The housing 102 is operable to retain a sample of amniotic fluid. Exemplary dimensions of the syringe 100 are 8″ long by 1″ wide.

The syringe includes a means for drawing the amniotic sac into the recessed portion 105 of the insertion end 104. For example, a plunger 108 disposed within and sealingly engaged with the tubular housing 102 may be used. The plunger 108 is operable to slide from the insertion end 104 of the housing 102 to the handle end 106 of the housing 102. When this occurs, the plunger 108 operates to draw a portion of the amniotic sac into the recessed portion 105 of the insertion end 104, until the amniotic sac contacts the puncture member 110. While the amniotic sac is pulled into the recessed portion 105 of the insertion end 104, a closed environment is created between the fluid opening of the recessed portion and the amniotic sac. In this state, the puncture member 110 is operates to pierce the amniotic sac, allowing amniotic fluid to flow unencumbered into the fluid opening 107 of the insertion end 104. The plunger 108 then operates to pull the amniotic fluid through the fluid opening 107 and into the fluid receiving area 109 of the housing 102.

In one embodiment, the puncture member 110 includes one or more sharp or serrated edges disposed along the recessed portion 105 of the insertion end 104, as illustrated in FIGS. 2-6. For this embodiment, the puncture member 110 may be made of plastic.

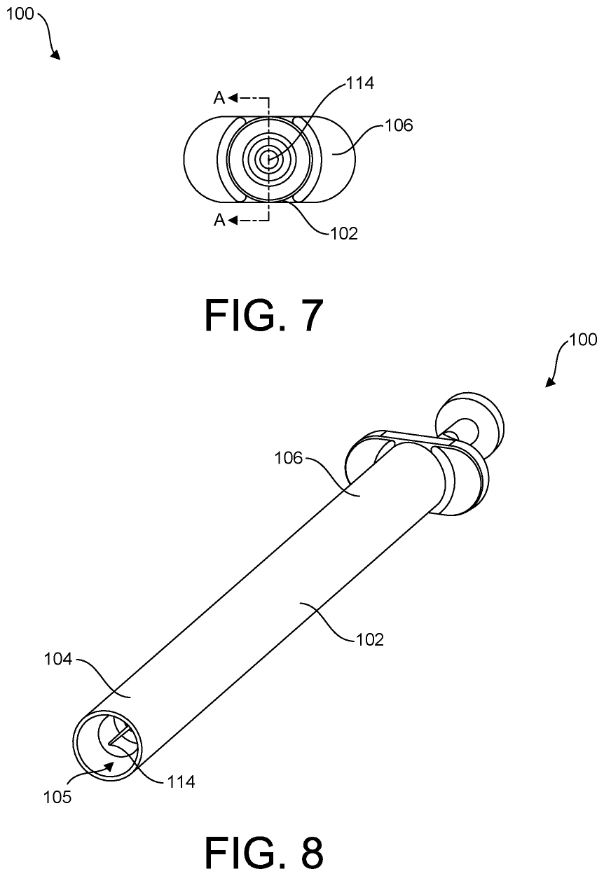

Turning to FIGS. 7-10, an alternative embodiment of the puncture member 110 is shown. Here, the puncture member resembles a needle 114 with a sharp point capable of puncturing the amniotic sac. The needle 114 may be made from plastic or metal, for example, and the for this embodiment, the fluid opening 107 is disposed within the needle point. The needle 114 is disposed within the recessed portion 105 of the insertion end 104 such that the needle point is sub-flush to the outer lip 111 of the syringe 100. In like fashion, the plunger 108 then operates to pull the amniotic fluid through the fluid opening 107 and into the fluid receiving area 109 of the housing 102 without rupturing the amniotic sac or inducing labor, enabling amniocentesis to be performed in a safe manner without risk to the fetus.

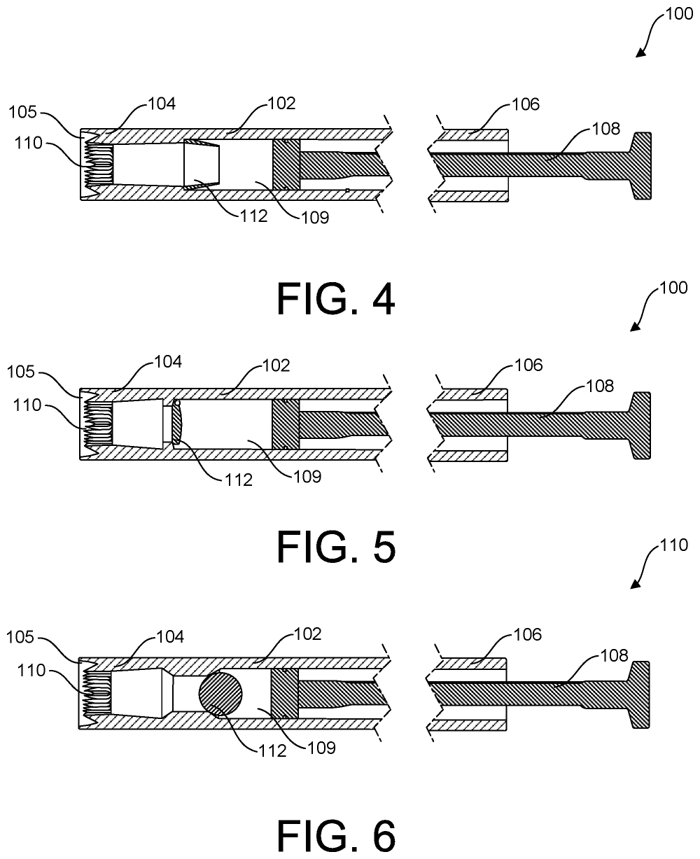

Any of the aforementioned embodiments may include a valve 112 operable to prevent amniotic fluid that has been drawn into the fluid receiving area 109 of the housing 102 from leaking back out of the housing 102, such as a check valve as illustrated in FIG. 4, a rocker valve as illustrated in FIG. 5, or a ball valve as illustrated in FIG. 6, for example.

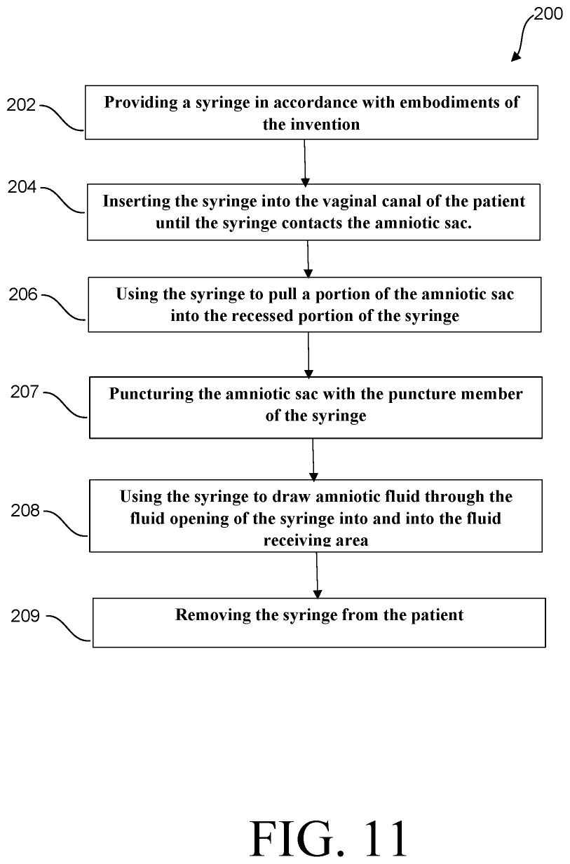

Turning to FIG. 11, a method 200 of rupturing an amniotic sac and obtaining a sample of amniotic fluid and inducing contractions in a patient is provided in accordance with embodiments of the invention. In step (a) 202, the syringe 100 is provided. In step (b) 204, the syringe 100 is inserted into the vaginal canal of the patient until the syringe 100 contacts the amniotic sac. In step (c) 206, the plunger 108 of the syringe 100 is actuated and pulled from the insertion end 104 of the syringe 100 towards the handle end 106, said pulling of the plunger 108 drawing in a portion of the amniotic sac into the recessed portion 105 of the insertion end 104 of housing 102 and then into contact with the puncture member 110. In step (d) 207, the puncture member thereby pierces the amniotic sac. In step (e) 208, the plunger 108 of the syringe 100 is pulled further away from the insertion end 104 in order to draw a sample of amniotic fluid through the fluid opening 107 and into the fluid receiving area 109 of housing 102. Because the sac is pulled into recessed portion 105 of the insertion end 104 of housing 102 prior to puncture, the puncture causes no lacerations to the fetus. In step (f) 209, the syringe 100 is removed from the patient, wherein the syringe 100 contains a pure sample of the amniotic fluid without interacting with the body fluid of the patient. The pure fluid can be tested for pre-birth complications or defects.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A syringe for rupturing an amniotic sac and obtaining a sample of amniotic fluid, said syringe comprising:
a housing having an open insertion end opposite a handle end, wherein said open insertion end comprises a recessed portion disposed within said open insertion end, wherein said recessed portion comprises a fluid opening;
a puncture member disposed in the recessed portion of the open insertion end;
a plunger disposed within and sealingly engaged with the tubular housing, said plunger operable to slide from the insertion end of the housing to the handle end of the housing,
wherein a portion of the housing between the plunger and the fluid opening defines a fluid receiving area,
wherein the fluid opening of the open insertion end is in fluid communication with the fluid receiving area,
wherein the plunger is operable to draw a portion of the amniotic sac into the recessed portion of the open insertion end,
wherein the puncture member is operable to pierce the amniotic sac, and
wherein said plunger is operable to draw in a sample of amniotic fluid from the amniotic sac into the fluid receiving area.

2. The syringe of claim 1, wherein the puncture member comprises one or more serrated edges.

3. The syringe of claim 1, wherein the fluid opening is disposed within the puncture member.

4. The syringe of claim 3, wherein the puncture member is a needle with the fluid opening disposed at an end of the needle opposite the plunger.

5. The syringe of claim 1 comprising a valve operable to prevent amniotic fluid that has been drawn into the fluid receiving area from leaking out the fluid opening of the open insertion end.

6. A method of rupturing an amniotic sac and obtaining a sample of amniotic fluid and inducing contractions in a patient, comprising:
(a) providing a syringe, comprising:
a housing having an open insertion end opposite a handle end, wherein said open insertion end comprises a recessed portion disposed within said open insertion end, wherein said recessed portion comprises a fluid opening;
a puncture member disposed in the recessed portion of the open insertion end;
a plunger disposed within and sealingly engaged with the tubular housing, said plunger operable to slide from the insertion end of the housing to the handle end of the housing,
wherein a portion of the housing between the plunger and the fluid opening within the open insertion end defines a fluid receiving area,
wherein the fluid opening of the open insertion end is in fluid communication with the fluid receiving area,
wherein the plunger is operable to draw a portion of the amniotic sac into the recessed portion of the open insertion end,
wherein the puncture member is operable to pierce the amniotic sac, and
wherein said plunger is operable to draw in a sample of amniotic fluid from the amniotic sac into the fluid receiving area.
(b) inserting the syringe into the vaginal canal of the patient until the syringe contacts the amniotic sac;
(c) pulling the handle of the plunger away from the insertion end of the syringe until a portion of the amniotic sac is drawn into the recessed portion of the open insertion end;
(d) piercing the amniotic sac with the puncture member;
(e) pulling the handle of the plunger further away from the insertion end of the syringe until an appropriate amount of amniotic fluid is collected in the fluid receiving area; and
(f) removing the syringe from the patient, wherein the fluid receiving area contains a pure sample of the amniotic fluid.

7. The syringe of claim 6, wherein the puncture member of the syringe comprises a serrated edge.

8. The syringe of claim 6, wherein the fluid opening is disposed within the puncture member.

9. The syringe of claim 8, wherein the puncture member is a needle with the fluid opening disposed at an end of the needle opposite the plunger.

10. The syringe of claim 6 comprising a valve operable to prevent amniotic fluid that has been drawn into the fluid receiving area from leaking out the fluid opening of the open insertion end.

11. A syringe for rupturing an amniotic sac and obtaining a sample of amniotic fluid, said syringe comprising:
a housing having a recessed open insertion end opposite a handle end with a fluid receiving area between said open insertion end and said handle end, wherein said open insertion end is in fluid communication with said fluid receiving area;
a puncture member disposed within said recess of said open insertion end, wherein said recessed portion comprises a fluid opening; and
a means for drawing fluid through the fluid opening into the fluid receiving area.

12. The syringe of claim 11, wherein the puncture member comprises a serrated edge.

13. The syringe of claim 11, wherein the fluid opening is disposed within the puncture member.

14. The syringe of claim 13, wherein the puncture member is a needle with the fluid opening disposed at an end of the needle.

15. The syringe of claim 11 comprising a valve operable to prevent amniotic fluid that has been drawn into the fluid receiving area from leaking out the fluid opening of the open insertion end.
